(19) United States
(12) Patent Application Publication
(10) Pub. No.: US 2008/0039466 A1
(43) Pub. Date: Feb. 14, 2008

(54) 2-(3-Substituted-Aryl) Amino-4-Aryl-Thiazoles As Tyrosine Kinase Inhibitors

(75) Inventors: Alain Moussy, Paris (FR); Marco Ciufolini, Lyon (FR); Camille Wermuth, Strasbourg (FR); Bruno Giethlen, Illkirch (FR)

Correspondence Address: FOLEY AND LARDNER LLP;SUITE 500, 3000 K STREET NW, WASHINGTON, DC, 20007, US

(73) Assignee: AB Science

(21) Appl. No.: 10/587,436

(22) PCT Filed: Jan. 28, 2005
(86) PCT No.: PCT/IB05/00401

§ 371 Date: Jul. 2, 2007

Related U.S. Application Data
(60) Provisional application No. 60/540,064, filed on Jan. 30, 2004.

(51) Int. Cl. A61K 31/5377; A61K 31/426; A61K 31/496; A61P 37/00; C07D 295/00; C07D 471/12; C07D 277/02; A61P 35/00; A61K 31/505

(52) U.S. Cl. 514/236.8; 514/252.14; 514/253.1; 514/342; 514/370; 544/122; 544/124; 544/295; 544/364; 546/270.4; 548/190

(57) ABSTRACT

The present invention relates to novel compounds selected from 2-(3substitutedaryl)amino-4-aryl-thiazoles that selectively modulate, regulate, and/or inhibit signal transduction mediated by certain native and/or mutant tyrosine kinases implicated in a variety of human and animal diseases such as cell proliferative, metabolic, allergic, and degenerative disorders. More particularly, these compounds are potent and selective c-kit inhibitors.

The present invention relates to novel compounds selected from 2-(3-substitutedaryl)amino-4-aryl-thiazoles that selectively modulate, regulate, and/or inhibit signal transduction mediated by certain native and/or mutant tyrosine kinases implicated in a variety of human and animal diseases such as cell proliferative, metabolic, allergic, and degenerative disorders. More particularly, these compounds are potent and selective c-kit inhibitors.

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation.

As of today, there are about 58 known receptor tyrosine kinases. Other tyrosine kinases are the well-known VEGF receptors (Kim et al., Nature 362, pp. 841-844, 1993), PDGF receptors, c-kit and the FLK family. These receptors can transmit signals to other tyrosine kinases including Src, Raf, Frk, Btk, Csk, Abl, Fes/Fps, Fak, Jak, Ack. etc.

Among tyrosine kinase receptors, c-kit is of special interest. Indeed, c-kit is a key receptor activating mast cells, which have proved to be directly or indirectly implicated in numerous pathologies for which the Applicant filed WO 03/004007, WO 03/004006, WO 03/003006, WO 03/003004, WO 03/002114, WO 03/002109, WO 03/002108, WO 03/002107, WO 03/002106, WO 03/002105, WO 03/039550, WO 03/035050, WO 03/035049, U.S. 60/359,652 and U.S. 60/359,651.

It was found that mast cells present in tissues of patients are implicated in or contribute to the genesis of diseases such as autoimmune diseases (rheumatoid arthritis, inflammatory bowel diseases (IBD)) allergic diseases, tumor angiogenesis, inflammatory diseases, and interstitial cystitis. In these diseases, it has been shown that mast cells participate in the destruction of tissues by releasing a cocktail of different proteases and mediators such as histamine, neutral proteases, lipid-derived mediators (Prostaglandins, thromboxanes and leucotrienes), and various cytokines (IL-1, IL-2, IL-3, IL-4, IL-5, IL-6, IL-8, TNF-α, GM-CSF, MIP-1a, MIP-1b, MIP-2 and IFN-γ).

The c-kit receptor also can be constitutively activated by mutations leading to abnormal cell proliferation and development of diseases such as mastocytosis and various cancers.

For this reason, it has been proposed to target c-kit to deplete the mast cells responsible for these disorders.

The main objective underlying the present invention is therefore to find potent and selective compounds capable of inhibiting wild type and/or mutated c-kit.

Many different compounds have been described as tyrosine kinase inhibitors, for example, bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), selenoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrole-substituted indolinones (U.S. Pat. No. 5,792,783, EP 934 931, U.S. Pat. No. 5,834,504, U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

However, none of these compounds have been described as potent and selective inhibitors of c-kit or of the c-kit pathway.

In connection with our previous invention which is described in WO2004014903, we found that compounds corresponding to the 2-(3-aminoaryl)amino-4-aryl-thiazoles are potent and selective inhibitors of c-kit or c-kit pathway. These compounds are good candidates for treating diseases such as autoimmunes diseases, inflammatory diseases, cancer and mastocytosis.

We now have determined that other 2-(3-substitutedaryl)amino-4-aryl-thiazole derivatives display very strong inhibitory activity on several forms of c-kit.

### DESCRIPTION

Therefore, the present invention relates to compounds belonging to the 2-(3-ketoarylamino-4-aryl-thiazoles. These compounds are capable of selectively inhibiting signal transduction involving the tyrosine phosphokinase c-kit and mutant forms thereof. In a first embodiment, the invention is aimed at compounds of formula I, which may represent either free base forms of the substances or pharmaceutically acceptable salts thereof: 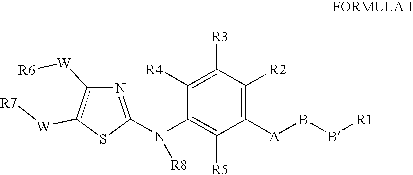
and wherein

R⁶ and R⁷ are independently from each other chosen from one of the following:

i) hydrogen, a halogen (selected from F, Cl, Br or I),

ii) an alkyl¹ group defined as a linear, branched or cycloalkyl group containing from 1 to 10 carbon atoms, or from 2 or 3 to 10 carbon atoms, (for example methyl, ethyl, propyl, butyl, pentyl, hexyl . . . ) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl;

(iii) an aryl¹ group defined as phenyl or a substituted variant thereof bearing any combination, at any one ring position, of one or more substituents such as

halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   (iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   (v) trifluoromethyl, carboxyl, cyano, nitro, formyl, hydroxy, N(alkyl¹)(alkyl¹), and amino, the latter nitrogen substituents optionally in the form of a basic nitrogen functionality.

R⁸ is one of the following:

(i) hydrogen, or

(ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or

(iii) CO—R⁸ or COOR⁸ or CONHR8 or SO2R8 wherein R8 may be

a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R2, R3, R4 and R5 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆ alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

A is: CH₂, 0, S, SO2, CO, or COO,

B is a bond or NH, NCH3, NR*, (CH2)n (n is 0, 1 or 2), O, S, SO2, CO, or COO,

B′ is a bond or NH, NCH3, NR*, (CH2)n (n is 0, 1 or 2), O, S, SO2, CO or COO;

R* being an alkyl¹, aryl¹ or heteroaryl¹

W is a bond or a linker selected from NH, NHCO, NHCOO, NHCONH, NHSO2, NHSO2NH, CO, CONH, COO, COCH2, (CH2)n (n is 0, 1 or 2), CH2-CO, CH2COO, CH2-NH, O, OCH2, S, SO2, and SO2NH

R¹ is:

a) a linear or branched alkyl group containing from 1 to 10 carbon atoms optionally substituted with at least one heteroatom, notably a halogen selected from I, Cl, Br and F, and/or bearing a pendant basic nitrogen functionality;

b) an aryl or heteroaryl group optionally substituted by an alkyl or aryl group optionally substituted with a heteroatom, notably a halogen selected from I, Cl, Br and F or bearing a pendant basic nitrogen functionality

c) an alkyl¹, aryl¹ or heteroaryl¹.

It will be understood that a C1-C10 alkyl encompasses a methyl, ethyl, propyl, and a C2 to C4 alkyl or a C2 to C10 alkyl.

For example, a subset of compounds may correspond to 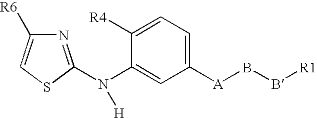
Wherein R1, R4 and R6 have the meaning as defined above.

It will be understood that A-B-B′ includes but is not limited to:

CH2, CH2-CO, CH2-CO—CH2, CH2COO, CH2-CH2-CO, CH2-CH2-COO, CH2-NH, CH2-CH2-NH, CH2-NH—CH2 or CH2-NH—CO or CH2-CO—NH

It will be understood that A-B-B′ also includes but is not limited to:

CO—CH2, COO—CH2, CO—CH2-CH2, CO—NH, or CO—NH—CH2 as well as O—CH2

It will also be understood that NH in B or B′ can also be NCH3

In the above formula I, when W is other than a single bond, it will be understood that A can be also be NH or NCH3.

In the above formula, the following combinations are contemplated:
R6 is (iv), R4 is H or CH3, A-B-B′ is CO—NH and R1 is as defined above.
R6 is (iv), R4 is H or CH3, A-B-B′ is CH2-CO—NH and R1 is as defined above.
R6 is (iv), R4 is H or CH3, A-B-B′ is CH2-CO and R1 is as defined above.
R6 is (iv), R4 is H or CH3, A-B-B′ is CH2-NH—CO and R1 is as defined above.
R6 is (iv), R4 is H or CH3, A-B-B′ is CH2-NH and R1 is as defined above.
R6 is (iv), R4 is H or CH3, A-B-B′ is CH2 and R1 is as defined above.
R6 is W-(iv), R4 is a C₁-C₂ alkyl, A-B-B′ is CO—NH and R1 is as defined above.
R6 is (iv), R4 is a C₁-C₂ alkyl, A-B-B′ is CH2-CO—NH and R1 is as defined above.
R6 is (iv), R4 is a C₁-C₂ alkyl, A-B-B′ is CH2-CO and R1 is as defined above.
R6 is a pyridyl according to (iv), R4 is a C₁-C₂ alkyl, A-B-B′ is CO—NH, CH2-CO—NH, CH2-CO, CH2-NH, CH2-NH—CO and R1 is as defined above.

In the above combination, R1 can be an alkyl¹.

In the above combination, R1 can be an aryl¹.

In the above combination, R1 can be an heteroaryl¹.

In one preferred embodiment, when ABB′ is CONH, the invention is directed to compounds of the following formula I-1:

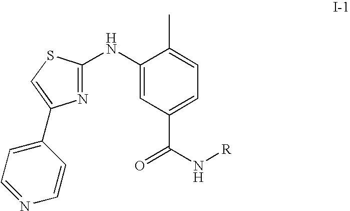

wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted by an alkyl, a cycloalkyl, an aryl or heteroaryl group optionally substituted with a heteroatom, notably a halogen selected from I, Cl, Br and F and/or bearing a pendant basic nitrogen functionality.

In one other preferred embodiment, the invention is directed to amide-aniline compounds of the following formula I-2:

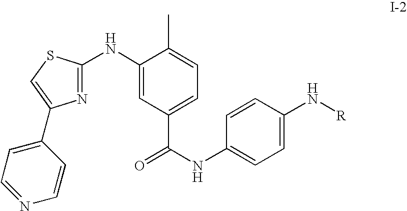

wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with a heteroatom, notably a halogen selected from I, Cl, Br and F and/or bearing a pendant basic nitrogen functionality; or a a cycloalkyl, an aryl or heteroaryl group optionally substituted with a cycloalkyl, an aryl or heteroaryl group optionally substituted with:
a heteroatom, notably a halogen selected from I, Cl, Br and F and/or bearing a pendant basic nitrogen functionality;
a SO2-R group wherein R is an alkyl, cycloalkyl, aryl or heteroaryl optionally substituted with an heteroatom, notably a halogen selected from I, Cl, Br and F and/or bearing a pendant basic nitrogen functionality;
a CO—R or a CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Among the particular compounds in which R1 has the meaning as depicted above, the invention is directed to amide-benzylamine compounds of the following formula I-3:

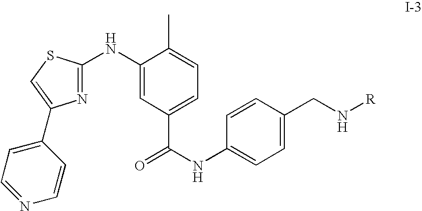

wherein R is H or an organic group that can be selected for example from a linear or branched alkyl group containing from 1 to 10 carbon atoms optionally substituted with at least one heteroatom, notably a halogen selected from I, Cl, Br and F, and/or bearing a pendant basic nitrogen functionality; a cycloalkyl, aryl or heteroaryl group optionally substituted with an heteroatom, notably a halogen selected from I, Cl, Br and F or bearing a pendant basic nitrogen functionality; or an alkyl, cycloalkyl, aryl or heteroaryl group substituted by a alkyl, cycloalkyl, aryl or heteroaryl group optionally substituted with a heteroatom, notably a halogen selected from I, Cl, Br and F or bearing a pendant basic nitrogen functionality;

a —SO2-R group wherein R is an alkyl, cycloalkyl, aryl or heteroaryl group optionally substituted with an heteroatom, notably a halogen selected from I, Cl, Br and F or bearing a pendant basic nitrogen functionality; or a —CO—R or a —CO—NRR′ group, wherein R and R′ are independently chosen from H or an aryl heteroaryl, alkyl and cycloalkyl group optionally substituted with at least one heteroatom and/or bearing a pendant basic nitrogen functionality.

Among the particular compounds in which R1 has the meaning as depicted above, the invention is directed to amide-phenol compounds of the following formula I-4:

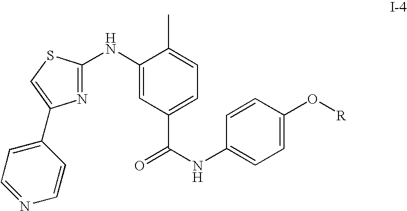

wherein R is H or an organic group that can be selected for example from a linear or branched alkyl group containing from 1 to 10 carbon atoms optionally substituted with at least one heteroatom, notably a halogen selected from I, Cl, Br and F, and/or bearing a pendant basic nitrogen functionality;

a cycloalkyl, aryl or heteroaryl group optionally substituted with a heteroatom, notably a halogen selected from I, Cl, Br and F and/or bearing a pendant basic nitrogen functionality; or an alkyl, cycloalkyl, aryl or heteroaryl group substituted by a alkyl, cycloalkyl, aryl or heteroaryl group optionally substituted with a heteroatom, notably a halogen selected from I, Cl, Br and F and/or bearing a pendant basic nitrogen functionality;

a —SO2-R group wherein R is an alkyl, cycloalkyl, aryl or heteroaryl group optionally substituted with an heteroatom, notably a halogen selected from I, Cl, Br and F and/or bearing a pendant basic nitrogen functionality; or a —CO—R or a —CO—NRR′ group, wherein R and R′ are independently chosen from H or an aryl, heteroaryl, alkyl and cycloalkyl group optionally substituted with at least one heteroatom and/or bearing a pendant basic nitrogen functionality.

Among compounds of formula I, the invention is particularly directed to 3-(thiazol-2-ylamino)-benzamide compounds of the following formula I-5:

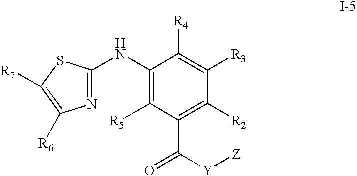

wherein Y is a single bond, a linear or branched alkyl group containing from 1 to 10 carbon atoms, especially CH2 or CH2-CH2; or NH

wherein Z represents an aryl or heteroaryl group, optionally substituted at one or more ring position with any permutation of the following groups:
a halogen such as F, Cl, Br, I;
a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an O—R, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaRb, where Ra and Rb represents a hydrogen, or a linear or branched alkyl group containing from 1 to 10 carbon atoms optionally substituted with at least one heteroatom (for example a halogen) and/or bearing a pendant basic nitrogen functionality or a cycle; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;

R² is hydrogen, halogen or a linear or branched alkyl group containing from 1 to 10 carbon atoms, trifluoromethyl, cyano or alkoxy;

R³ is hydrogen, halogen or a linear or branched alkyl group containing from 1 to 10 carbon atoms, trifluoromethyl, cyano or alkoxy;

R⁴ is hydrogen, halogen or a linear or branched alkyl group containing from 1 to 10 carbon atoms, trifluoromethyl, cyano or alkoxy;

R⁵ is hydrogen, halogen or a linear or branched alkyl group containing from 1 to 10 carbon atoms, trifluoromethyl, cyano or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality; and R⁷ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, an halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

An example of preferred compounds of the above formula is depicted below:

### 001: 4-[4-Methyl-3-(4-pyridin-4-yl-thiazol-2-ylamino)-benzoylamino]-benzoic acid 2-diethylamino-ethyl ester

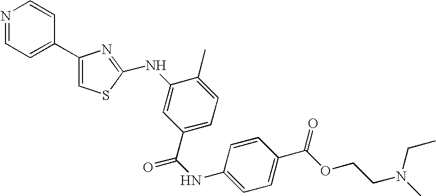

Among the compounds of formula I, the invention is particularly embodied by the compounds of the following formula II: 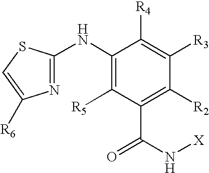
Formula II

wherein X is R or NRR′ and wherein R and R′ are independently chosen from H, an aryl, a heteroaryl, an allyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,

R² is hydrogen, halogen or a linear or branched alkyl group containing from 1 to 10 carbon atoms, trifluoromethyl or alkoxy;

R³ is hydrogen, halogen or a linear or branched alkyl group containing from 1 to 10 carbon atoms, trifluoromethyl or alkoxy;

R⁴ is hydrogen, halogen or a linear or branched alkyl group containing from 1 to 10 carbon atoms, trifluoromethyl or alkoxy;

R⁵ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R⁶, which in the formula II is connected to position 4 of the thiazole ring, may instead occupy position 5 of the thiazole ring.

Among the preferred compounds corresponding formulas I and II, the invention is directed to compounds in which R1 and X, respectively, is a substituted alkyl, aryl or heteroaryl group bearing a pendant basic nitrogen functionality represented for example by the structures a to m shown below, wherein the wavy line and the arrow line correspond to the point of attachment to core structure of formula I or II.

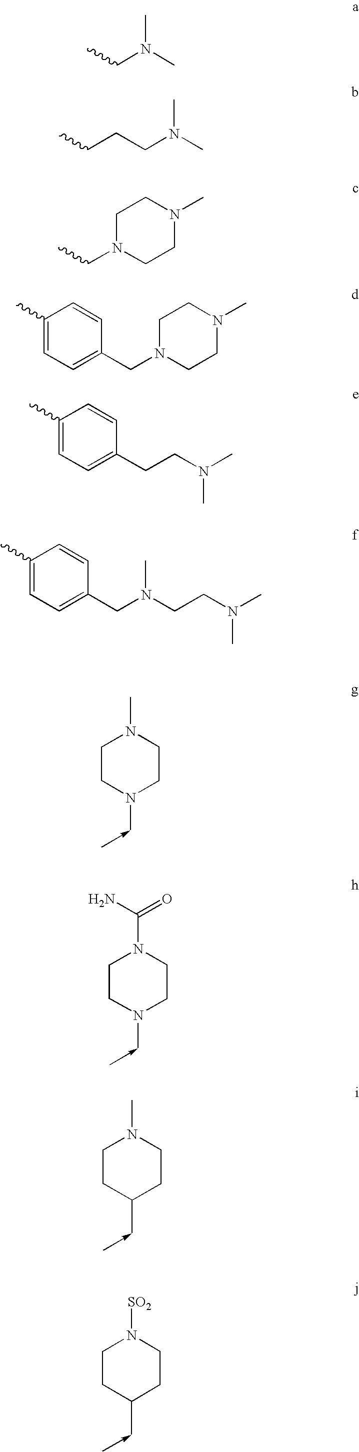

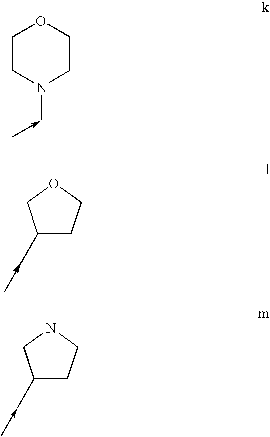

Among group a to f and g to m R1 of formula I and X of formula II is preferentially group d. Also, for g to m, the arrow includes a point of attachment to the core structure via a phenyl group.

Furthermore, among the preferred compounds of formula I or II, the invention concerns the compounds in which R² and R³ are hydrogen. Preferentially, R⁴ is a methyl group and R⁵ is H. In addition, R⁶ is preferentially a 3-pyridyl group (cf. structure g below), or a 4-pyridyl group (cf. structure h below). The wavy line in structure g and h correspond to the point of attachment to the core structure of formula I or II.

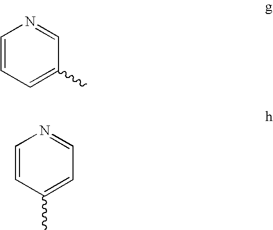

Thus, the invention contemplates:
1—A compound of formula II as depicted above, wherein X is group d and R⁶ is a 3-pyridyl group.
2—A compound of formula II as depicted above, wherein X is group d and R⁴ is a methyl group.
3—A compound of formula I or II as depicted above, wherein R¹ is group d and R² is H.
4—A compound of formula I or II as depicted above, wherein R¹ is group d and R³ is H.
5—A compound of formula I or II as depicted above, wherein R¹ is group d and R² and/or R³ and/or R⁵ is H.
6—A compound of formula I or II as depicted above, wherein R⁶ is a 3-pyridyl group and R³ is a methyl group.
7—A compound of formula I or II as depicted above, wherein R⁶ is a 3-pyridyl group and R² is H.
8—A compound of formula I or II as depicted above, wherein R² and/or R³ and/or R⁵ is H and R⁴ is a methyl group.
9—A compound of formula I or II as depicted above wherein R² and/or R³ and/or R⁵ is H, R⁴ is a methyl group and R⁶ is a 3-pyridyl group.

Among the compounds of formula II, the invention is particularly embodied by the compounds wherein R², R³, R⁵ are hydrogen, corresponding to the following formula II-1:

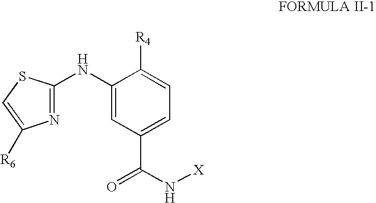

wherein X is R or NRR′ and wherein R and R′ are independently chosen from H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with an heteroatom, notably a halogen selected from I, Cl, Br and F or bearing a pendant basic nitrogen functionality; or a a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula II is connected to position 4 of the thiazole ring, may instead occupy position 5 of the thiazole ring.

### EXAMPLES

### 002: N-(3,5-Bis-trifluoromethyl-phenyl)-4-methyl-3-(4-pyridin-4-yl-thiazol-2-ylamino)-benzamide

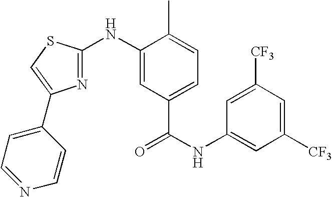

¹H NMR (DMSO-d⁶) δ=2.36 (s, 3H, ArCH₃); 7.43 (d, 1H, J=7.5 Hz, Ar—H); 7.68 (dd, 1H, J=7.5, 1.5 Hz, Ar—H); 7.73 (s, 1H, thiazol-H); 7.82 (m, 3H, pyridyl-H+ Ar—H); 8.54 (m, 4H, pyridyl-H+2×Ar—H); 8.85 (br s, 1H, Ar—H); 9.67 (s, 1H, NH), 10.84 (s, 1H, NH).

### 003: N-(3,5-Bis-trifluoromethyl-phenyl)-4-methyl-3-(4-pyridin-3-yl-thiazol-2-ylamino)-benzamide

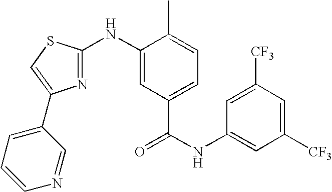

### 092: N-Cyclohexyl-4-methyl-3-(4-pyridin-4-yl-thiazol-2-ylamino)-benzamide

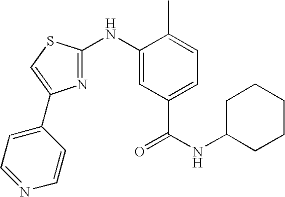

¹H NMR (DMSO-d⁶) δ=1.00-1.40 (m, 5H, cyclo-H); 1.50-1.85 (m, 5H, cyclo-H); 2.34 (s, 3H, ArCH₃); 7.28 (d, 1H, J=7.9 Hz, Ar—H); 7.48 (dd, 1H, J=7.9, 1.5 Hz, Ar—H); 7.67 (s, 1H, thiazol-H); 7.82 (d, 2H, J=6.0 Hz, pyridyl-H); 8.57 (d, 2H, J=6.0 Hz, pyridyl-H); 8.63 (d, 1H, J=1.5 Hz, Ar—H); 9.55 (s, 1H, NH).

### 093: 4-Methyl-N-(1-methyl-1-indol-6-yl)-3-(4-pyridin-3-yl-thiazol-2-ylamino)-benzamide

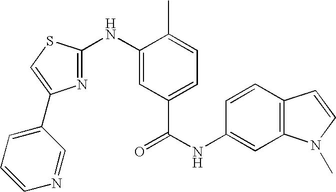

### 094: N-(2-Methoxy-ethyl)-4-methyl-3-(4-pyridin-4-yl-thiazol-2-ylamino)-benzamide

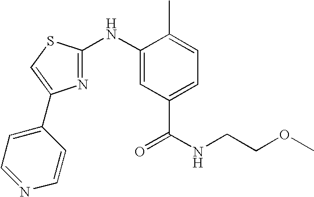

### 096: N-(2-Cyano-ethyl)-4-methyl-3-(4-pyridin-4-yl-thiazol-2-ylamino)-benzamide

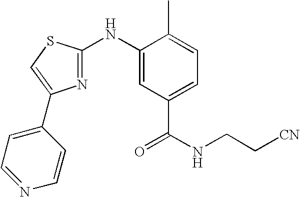

Among the compounds of formula II, the invention is particularly embodied by the compounds wherein X is a -substituted Aryl group, corresponding to the N-[3-(Thiazol-2-ylamino)-phenyl]-amide family and the following formula II-3:

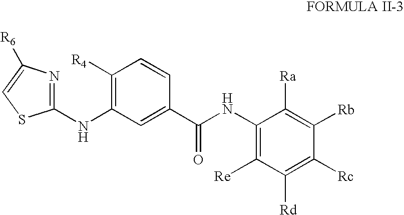

wherein Ra, Rb, Rc, Rd, Re are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;

a —SO2-R group wherein R is an alkyl, cycloalkyl, aryl or heteroaryl optionally substituted with a heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and or bearing a pendant basic nitrogen functionality;

Ra, Rb, Rc, Rd, Re may also be
a halogen such as I, Cl, Br and F
a NRR′ group where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
an OR group where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality; a —SO2-R′ group wherein R′ is an alkyl, cycloalkyl, aryl or heteroaryl optionally substituted with a heteroatom, notably a halogen selected from I, Cl, Br and F or bearing a pendant basic nitrogen functionality;
a NRaCORb group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a CN group
a trifluoromethyl group

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 028: N-(2-Fluoro-3-trifluoromethyl-phenyl)-4-methyl-3-(4-pyridin-4-yl-thiazol-2-ylamino)-benzamide

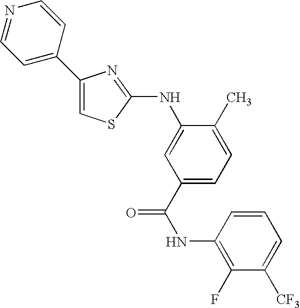

### 029: N-(3-Fluoro-phenyl)-4-methyl-3-(4-pyridin-4-yl-thiazol-2-ylamino)-benzamide

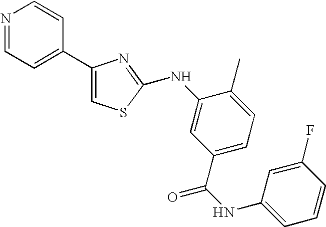

### 030: 4-Methyl-3-(4-pyridin-4-yl-thiazol-2-ylamino)-N-(3-trifluoromethyl-phenyl)-benzamide

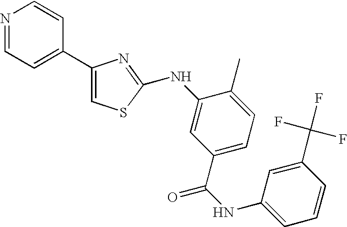

### 031: 4-Methyl-N-(4-methyl-3-trifluoromethyl-phenyl)-3-(4-pyridin-4-yl-thiazol-2-ylamino)-benzamide

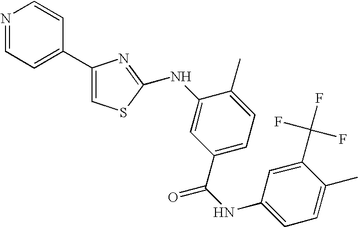

### 032: N-(2-Fluoro-5-trifluoromethyl-phenyl)-4-methyl-3-(4-pyridin-4-yl-thiazol-2-ylamino)-benzamide

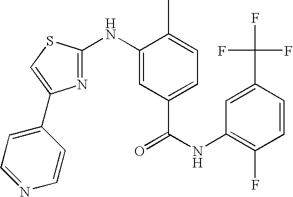

¹H NMR (DMSO-d⁶) δ=2.39 (s, 3H, ArCH₃); 7.41 (d, 1H, J=7.9 Hz, Ar—H); 7.54-7.70 (m, 3H, Ar—H); 7.72 (s, 1H, thiazol-H); 7.82 (d, 2H, J=6.0 Hz, pyridyl-H); 8.10 (dd, 1H, J=6.8, 2.2 Hz, Ar—H); 8.55 (d, 2H, J=6.0 Hz, pyridyl-H); 8.84 (d, 1H, J=1.8 Hz, Ar—H); 9.65 (s, 1H, NH); 10.31 (s, 1H, NH).

### 033: N-(4-Cyano-phenyl)-4-methyl-3-(4-pyridin-4-yl-thiazol-2-ylamino)-benzamide

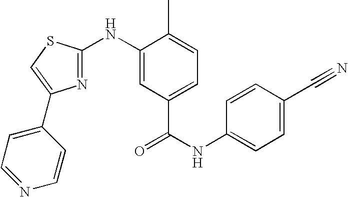

### 034: N-(4-Fluoro-phenyl-4-methyl-3-(4-pyridin-4-yl-thiazol-2-ylamino)-benzamide

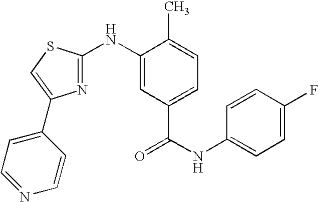

### 035: N-(3-Fluoro-4-methyl-phenyl)-4-methyl-3-(4-pyridin-4-yl-thiazol-2-ylamino)-benzamide

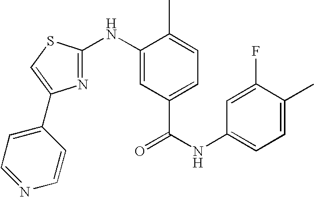

### 036: N-(4-tert-Butyl-phenyl)-4-methyl-3-(4-pyridin-4-yl-thiazol-2-ylamino)-benzamide

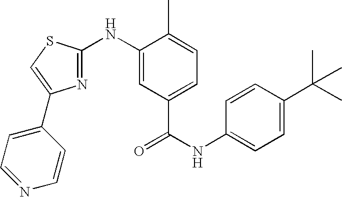

### 038: N-(3-Cyano-phenyl)-4-methyl-3-(4-pyridin-4-yl-thiazol-2-ylamino)-benzamide

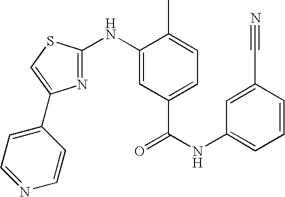

### 039: N-(3-Cyano-4-methyl-phenyl)-4-methyl-3-(4-pyridin-4-yl-thiazol-2-ylamino)-benzamide

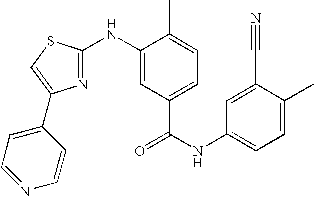

¹H NMR (DMSO-d⁶) δ=2.37 (s, 3H, ArCH₃); 2.46 (s, 3H, ArCH₃); 7.43 (m, 2H, Ar—H); 7.63 (dd, 1H, J=7.9, 1.8 Hz, Ar—H); 7.72 (s, 1H, thiazol-H); 7.83 (d, 2H, J=6.0 Hz, pyridyl-H); 7.96 (dd, 1H, J=8.3, 1.8 Hz, Ar—H); 8.19 (d, 1H, J=2.3 Hz, Ar—H); 8.55 (d, 2H, J=6.0 Hz, pyridyl-H); 8.81 (d, 1H, J=1.5 Hz, Ar—H); 9.65 (s, 1H, NH); 10.46 (s, 1H, NH).

### 040: N-(3-Bromo-phenyl)-4-methyl-3-(4-pyridin-4-yl-thiazol-2-ylamino)-benzamide

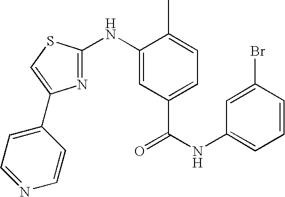

### 041: N-(3-Bromo-4-methyl-phenyl)-4-methyl-3-(4-pyridin-4-yl-thiazol-2-ylamino)-benzamide

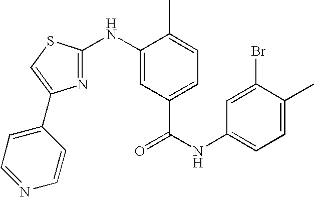

### 042: N-(3,5-Dibromo-4-methyl-phenyl)-4-methyl-3-(4-pyridin-4-yl-thiazol-2-ylamino)-benzamide

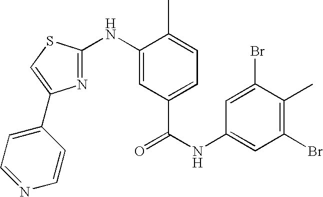

### 043: N-(3-Chloro-phenyl)-4-methyl-3-(4-pyridin-4-yl-thiazol-2-ylamino)-benzamide

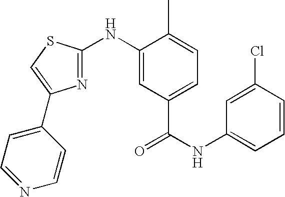

### 044: N-(3-Chloro-4-methyl-phenyl)-4-methyl-3-(4-pyridin-4-yl-thiazol-2-ylamino)-benzamide

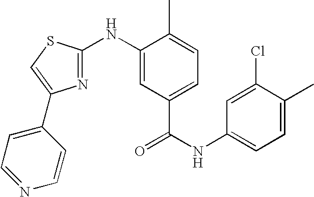

### 045: N-(3-Methoxy-phenyl)-4-methyl-3-(4-pyridin-4-yl-thiazol-2-ylamino)-benzamide

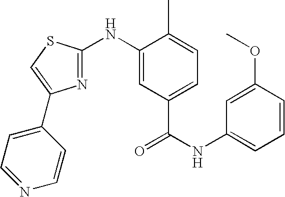

### 046: 4-Methyl-3-(4-pyridin-4-yl-thiazol-2-ylamino)-N-m-tolyl-benzamide

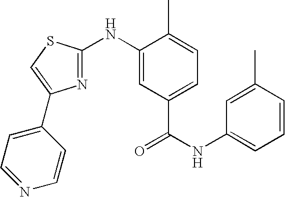

### 047: N-(4-Fluoro-3-methyl-phenyl)-4-methyl-3-(4-pyridin-4-yl-thiazol-2-ylamino)-benzamide

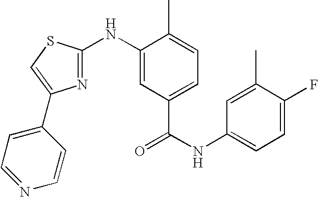

### 048: N-(3-Iodo-4-methyl)-4-methyl-3-(4-pyridin-4-yl-thiazol-2-ylamino)-benzamide

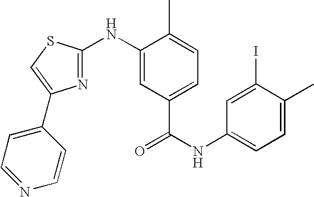

### 049: 4-Methyl-N-(3-nitro-phenyl)-3-(4-pyridin-4-yl-thiazol-2-ylamino)-benzamide

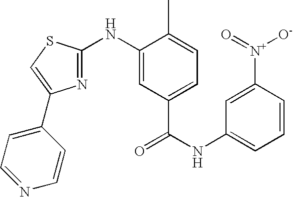

### 050: 4-Methyl-3-(4-pyridin-4-yl-thiazol-2-ylamino)-N-p-tolyl-benzamide

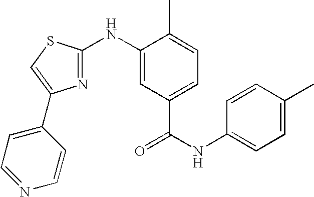

### 051: 4-Methyl-N-phenyl-3-(4-pyridin-4-yl-thiazol-2-ylamino)-benzamide

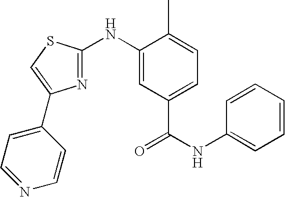

### 052: N-(3,4-Dimethyl-phenyl)-4-methyl-3-(4-pyridin-4-yl-thiazol-2-ylamino)-benzamide

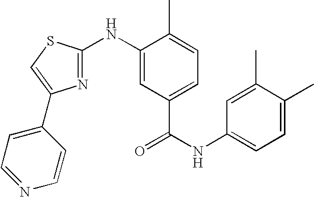

### 053: 4-Methyl-3-(4-pyridin-4-yl-thiazol-2-ylamino)-N-(3-trifluoromethoxy-phenyl)-benzamide

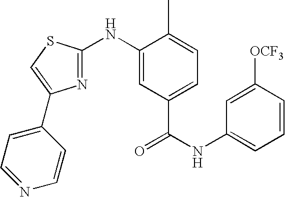

### 054: N-(3,4-Dicyano-phenyl)-4-methyl-3-(4-pyridin-4-yl-thiazol-2-ylamino)-benzamide

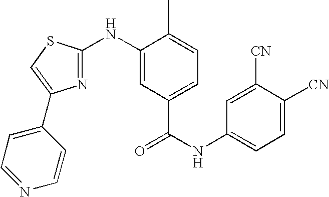

### 055: N-(2-Fluoro-5-methyl-phenyl)-4-methyl-3-(4-pyridin-4-yl-thiazol-2-ylamino)-benzamide

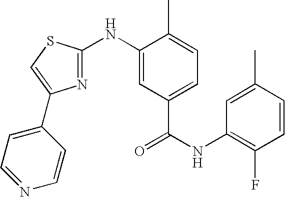

### 056: N-(2,4-Difluoro-phenyl)-4-methyl-3-(4-pyridin-4-yl-thiazol-2-ylamino)-benzamide benzamide

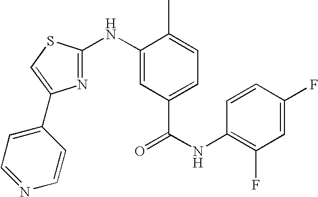

### 057: N-(4-Cyano-2-fluoro-phenyl)-4-methyl-3-(4-pyridin-4-yl-thiazol-2-ylamino)-benzamide

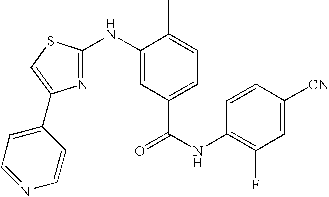

### 058: N-(2-Fluoro-4-methyl-phenyl)-4-methyl-3-(4-pyridin-4-yl-thiazol-2-ylamino)-benzamide

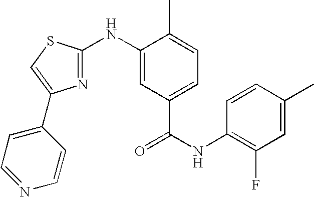

### 059: N-(2,4-Difluoro-phenyl)-4-methyl-3-(4-pyridin-3-yl-thiazol-2-ylamino)-benzamide

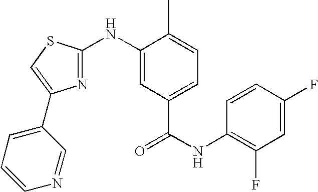

### 060: N-(4-Cyano-2-fluoro-phenyl)-4-methyl-3-(4-pyridin-3-yl-thiazol-2-ylamino)-benzamide

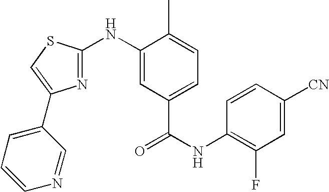

### 061: N-(2-Fluoro-4-methyl-phenyl)-4-methyl-3-(4-pyridin-3-yl-thiazol-2-ylamino)-benzamide

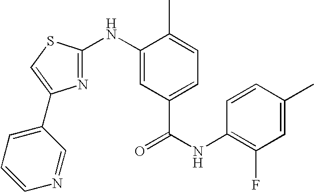

### 062: N-(4-Cyano-phenyl)-4-methyl-3-(4-pyridin-3-yl-thiazol-2-ylamino)-benzamide

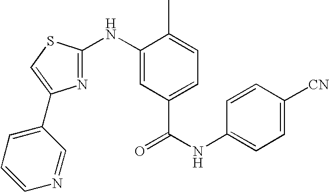

### 065: N-(4-Fluoro-phenyl)-4-methyl-3-(4-pyridin-3-yl-thiazol-2-ylamino)-benzamide

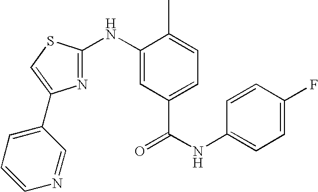

### 099: 4-Methyl-3-(4-pyridin-3-yl-thiazol-2-ylamino)-N-m-tolyl-benzamide

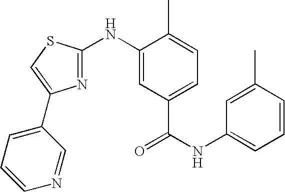

### 100: 4-Methyl-3-(4-pyridin-3-yl-thiazol-2-ylamino)-N-(3-trifluoromethyl-phenyl)-benzamide

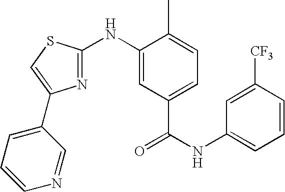

### 101: 4-Methyl-N-(4-methyl-3-trifluoromethyl-phenyl)-3-(4-pyridin-3-yl-thiazol-2-ylamino)-benzamide

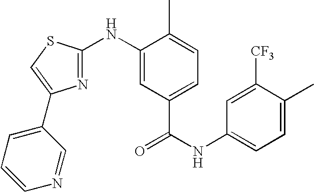

### 102: N-(2-Fluoro-3-trifluoromethyl-phenyl)-4-methyl-3-(4-pyridin-3-yl-thiazol-2-ylamino)-benzamide

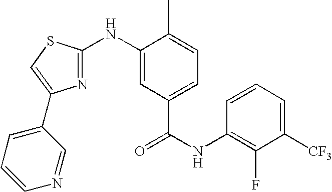

### 105: N-(4-Cyano-3-trifluoromethyl-phenyl)-4-methyl-3-(4-pyridin-3-yl-thiazol-2-ylamino)-benzamide

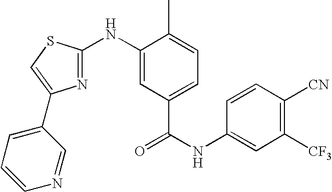

### 106: N-(4-Cyano-3-methyl-phenyl)-4-methyl-3-(4-pyridin-3-yl-thiazol-2-ylamino)-benzamide

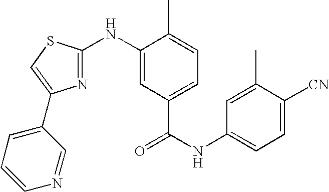

Among compounds of formula II, the invention is particularly embodied by the compounds wherein X is a -substituted-aryl group, corresponding to the 4-(4-substituted-1-ylmethyl)-N-[3-(thiazol-2-ylamino)-phenyl]-benzamide family and the following formula II-4: 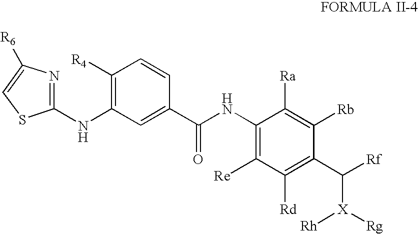
wherein X is a heteroatom, such as O or N

wherein Ra, Rb, Rd, Re, Rf, Rg, Rh are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a NRR′ group where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or an OR group where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality; a —SO2-R′ group wherein R′ is an alkyl, cycloalkyl, aryl or heteroaryl optionally substituted with a heteroatom, notably a halogen selected from I, Cl, Br and F or bearing a pendant basic nitrogen functionality;
or a NRaCORb group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Ra, Rb, Rd, Re can also be halogen such as Cl, F, Br, I or trifluoromethyl;

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 004: 4-Methyl-N-[4-(4-methyl-piperazin-1-ylmethyl)-3-trifluoromethyl-phenyl]-3-(4-pyridin-4-yl-thiazol-2-ylamino)-benzamide

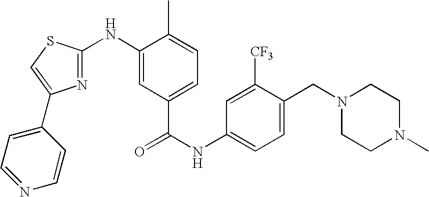

¹H NMR (MeOH-d₄) δ=2.41 (s, 6H, NCH₃+ArCH₃); 2.50-2.70 (m, 4H, pyperazine-H); 2.90 (m, 4H, pyperazine-H); 3.68 (br s, 2H, CH2-piperazine); 7.38 (d, 1H, J=7.9 Hz, Ar—H); 7.50 (m, 1H, thiazol-H); 7.60 (m, 1H, Ar—H); 7.76 (d, 1H, J=8.3 Hz, Ar—H); 7.90 (m, 2H, pyridyl-H); 8.00 (m, 1H, Ar—H); 8.12 (m, 1H, Ar—H); 8.46 (m, 2H, pyridyl-H); 8.90 (m, 1H, Ar—H).

### 005: 4-Methyl-N-{4-[1-(4-methyl-piperazin-1-yl)-ethyl]-phenyl}-3-(4-pyridin-3-yl-thiazol-2-ylamino)-benzamide

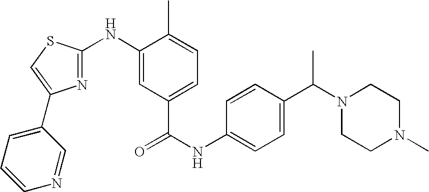

Among compounds of formula II, the invention is particularly embodied by the compounds wherein X is a -aryl-substituted group, corresponding to the 3-Disubstituted-amino-N-[3-(thiazol-2-ylamino)-phenyl]-benzamide family and the following formula II-5:

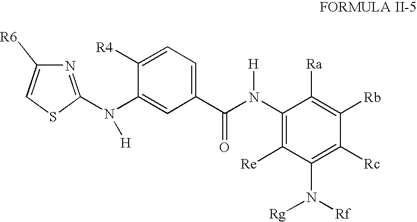

wherein Ra, Rb, Rc, Re, Rf, Rg are independently chosen from H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a NRR′ group where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or an OR group where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality; a —SO2-R′ group wherein R′ is an alkyl, cycloalkyl, aryl or heteroaryl optionally substituted with a heteroatom, notably a halogen selected from I, Cl, Br and F or bearing a pendant basic nitrogen functionality;
or a NRaCORb group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
or a COOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or a CONRaRb, where Ra and Rb are a hydrogen or a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or an NHCOOR, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or an NRaOSO₂Rb, where Ra and Rb are a linear or branched alkyl group containing from 1 to 10 carbon atoms atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
or a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Ra, Rb, Rc, Re can also be halogen such as Cl, F, Br, I or trifluoromethyl;

R⁴ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;

iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### EXAMPLES

### 089: N-(3-Dimethylamino-phenyl)-4-methyl-3-(4-pyridin-4-yl-thiazol-2-ylamino)-benzamide

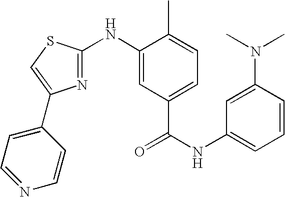

¹H NMR (DMSO-d⁶) δ=2.36 (s, 3H, ArCH₃); 2.88 (s, 6H, 2×CH₃); 6.50 (d, 1H, J=7.9 Hz, Ar—H); 7.10-7.30 (m, 3H, Ar—H); 7.38 (d, 1H, J=7.9 Hz, Ar—H); 7.62 (dd, 1H, J=7.9, 1.5 Hz, Ar—H); 7.70 (s, 1H, thiazol-H); 7.85 (d, 2H, J=6.4 Hz, pyridyl-H); 8.54 (d, 1H, J=6.4 Hz, pyridyl-H); 8.78 (br s, 1H, Ar—H); 9.63 (s, 1H, NH), 10.04 (s, 1H, NH).

### 090: N-(3-Dimethylamino-phenyl)-4-methyl-3-(4-pyridin-3-yl-thiazol-2-ylamino)-benzamide

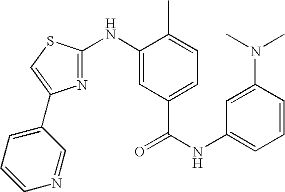

In a second embodiment, the invention is directed to a process for manufacturing a compound of formula I depicted above. This entails the condensation of a substrate of general formula 10 with a thiourea of the type 11.

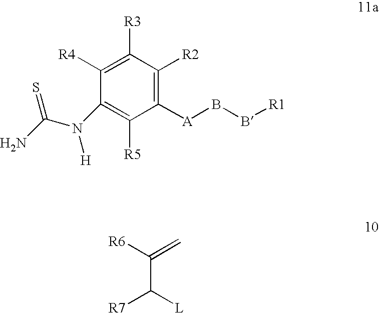

Substituent “L” in formula 10 is a nucleofugal leaving group in nucleophilic substitution reactions (for example, L can be selected from chloro, bromo, iodo, toluenesulfonyloxy, methanesulfonyloxy, trifluoromethanesulfonyloxy, etc., with L being preferentially a bromo group).

Group R1 in formula 11a corresponds to an alkoxy group.

The reaction of 10 with 1a-d leads to a thiozole-type product of formula 12a-d.

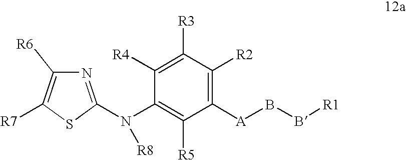

Formula 12a is the same as formula I. Therefore, R1 in 12a corresponds to R1 in formula I.

Examples of Compound Synthesis

General: All chemicals used were commercial reagent grade products. Dimethylformamide (DMF), methanol (MeOH) were of anhydrous commercial grade and were used without further purification. Dichloromethane and tetrahydrofuran (THF) were freshly distilled under a stream of argon before use. The progress of the reactions was monitored by thin layer chromatography using precoated silica gel 60F 254, Fluka TLC plates, which were visualized under UV light. Multiplicities in ¹H NMR spectra are indicated as singlet (s), broad singlet (br s), doublet (d), triplet (t), quadruplet (q), and multiplet (m) and the NMR spectrum were realized on a 300 MHz Bruker spectrometer.

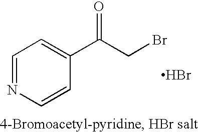

Dibromine (17.2 g, 108 mmol) was added dropwise to a cold (0° C.) solution of 4-acetyl-pyridine (12 g, 99 mmol) in acetic acid containing 33% of HBr (165 mL) under vigourous stirring. The vigorously stirred mixture was warmed to 40° C. for 2 h and then to 75° C. After 2 h at 75° C., the mixture was cooled and diluted with ether (400 mL) to precipitate the product. which was recovered by filtration and washed with ether and acetone to give white crystals (100%). This material may be recrystallised from methanol and ether.

¹H NMR (DMSO-d⁶) δ=5.09 (s, 2H, CH₂Br); 8.62 (m, 2H, pyridyl-H); 9.07 (m, 2H, pyridyl-H).

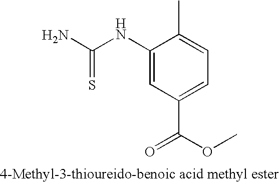

Benzoyl chloride (5.64 g, 80 mmol) was added dropwise to a well-stirred solution of ammonium thiocyanate (3.54 g, 88 mmol) in acetone (50 mL). The mixture was refluxed for 15 min, then, the 3-amino-4-methyl-benzoic acid methyl ester (13.2 g, 80 mmol) was added slowly portionswise. After 1 h, the reaction mixture was poured into water (350 mL) and the bright yellow precipitate was isolated by filtration. This crude solid was stirred at room temperature with an excess anhydrous potassium carbonate in 200 mL of methanol for 2 hours. Then, the solvent was removed under reduced pressure and the crude product wax extracted with ethyl acetate and washed with water. The organic layer was dried over Na₂SO₄ and concentrated to give a white solid. The solid was stirred in ether for 15 min and filtered to give the final product as a white solid.

¹H NMR (DMSO-d⁶) δ=2.22 (s, 3H, ArCH₃); 3.81 (s, 3H, CO₂CH₃); 7.38 (d, 1H, J=7.9 Hz, Ar—H); 7.70 (dd, 1H, J=7.9, 1.5 Hz, Ar—H); 7.82 (d, 1H, J=1.8 Hz, Ar—H).

### 4-Methyl-3-(4-pyridin-4-yl-thiazol-2-ylamino)-benzoic acid methyl ester

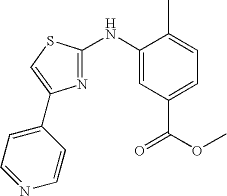

A mixture of 4-bromoacetyl-pyridine, HBr salt (0.40 g, 1.43 mmol), 4-methyl-3-thioureido-benzoic acid methyl ester (0.32 g, 1.43 mmol) and KHCO₃ (˜0.4 g) in ethanol (10 mL) was heated at 75° C. for 20 h. The mixture was cooled, filtered (removal of KHCO₃) and evaporated under reduced pressure. The residue was dissolved in CHCl₃ (40 mL) and washed with saturated aqueous sodium hydrogen carbonate solution and with water. The organic layer was dried over Na₂SO₄ and concentrated. The crude product was triturated in small amount of ethyl acetate and filtered to give the final product as an orange solid.

¹H NMR (DMSO-d⁶) δ=2.38 (s, 3H, ArCH₃); 3.88 (s, 3H, CO₂CH₃); 7.58 (dd, 1H, J=7.9, 1.8 Hz, Ar—H); 7.75 (s, 1H, thiazol-H); 7.85 (d, 2H, J=6.0 Hz, pyridyl-H); 8.62 (d, 1H, J=6.0 Hz, pyridyl-H); 9.12 (d, 1H, J=1.8 Hz, Ar—H); 9.63 (s, 1H, NH).

### N-(4-Cyano-phenyl)-4-methyl-3-(4-pyridin-4-yl-thiazol-2-ylamino)-benzamide

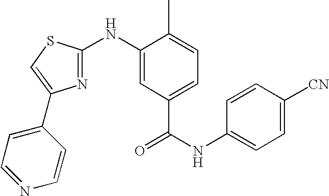

### 4-Methyl-3-(4-pyridin-4-yl-thiazol-2-ylamino)-benzoic acid methyl ester

A 2M solution of trimethyl aluminium in hexane (1.9 mL) was added dropwise to a cold (0° C.) solution of 4-amino-benzonitrile (0.29 g, 2.46 mmol) in anhydrous dichloromethane (30 mL) under argon atmosphere. The mixture was warmed to room temperature and stirred at room temperature for 30 min. A solution of 4-methyl-3-(4-pyridin-4-yl-thiazol-2-ylamino)-benzoic acid methyl ester (0.80 g, 2.46 mmol) in anhydrous dichloromethane (30 mL) and added slowly, and the resulting mixture was heated at reflux for 5 h. The mixture was cooled to 0° C. and quenched by dropwise addition of a 4N aqueous sodium hydroxide solution (3 mL). The mixture was extracted with dichloromethane (3×20 mL). The combined organic layers were washed with brine (3×20 mL) and dried over anhydrous MgSO₄. N-(4-Cyano-phenyl)-4-methyl-3-(4-pyridin-4-yl-thiazol-2-ylamino)-benzamide is obtained in 98% after trituration of the crude product in methanol.

¹H NMR (CDCl₃) δ=2.40 (s, 3H, ArCH₃); 7.40 (d, 1H, J=7.9 Hz, Ar—H); 7.63 (dd, 1H, J=7.9, 1.5 Hz, Ar—H); 7.72 (s, 1H, thiazole-H); 7.80-7.88 (m, 4H, Ar—H); 8.10 (d, 2H, J=8.6 Hz, Ar—H); 8.56 (m, 2H, Ar—H); 8.86 (d, 1H, J=1.8 Hz, Ar—H); 9.66 (br s, 1H, NH).

### EXAMPLES

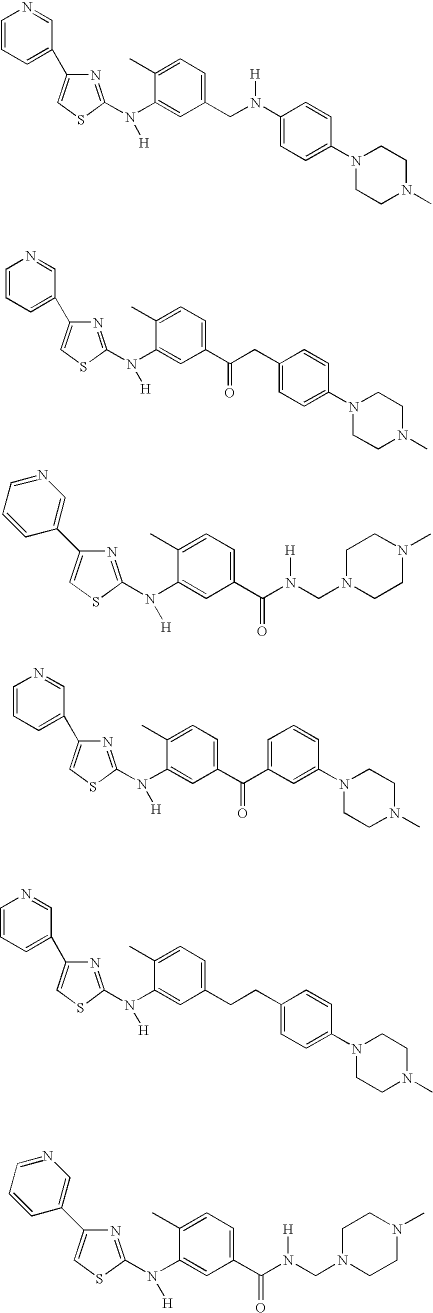

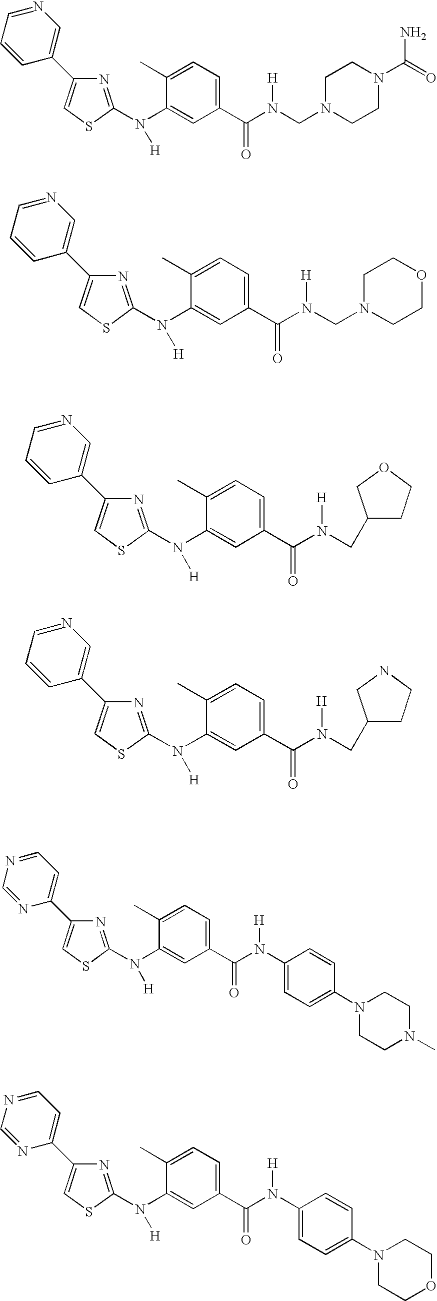

In a third embodiment, the invention relates to a pharmaceutical composition comprising a compound as depicted above.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

The composition of the invention can also take the form of a pharmaceutical or cosmetic composition for topical administration.

Such compositions may be presented in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the compound capable of depleting mast cells, such as a tyrosine kinase inhibitor, preferably a c-kit inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Permeability of Human Epidermis: An Electrometric Study,” J. Ivest. Dermatol., V.60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biology of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Berner, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

A second class of chemical enhancers are generally referred to as co-solvents. These materials are absorbed topically relatively easily, and, by a variety of mechanisms, achieve permeation enhancement for some drugs. Ethanol (Gale et. al., U.S. Pat. No. 4,615,699 and Campbell et. al., U.S. Pat. Nos. 4,460,372 and 4,379,454), dimethyl sulfoxide (U.S. Pat. Nos. 3,740,420 and 3,743,727, and U.S. Pat. No. 4,575,515), and glycerine derivatives (U.S. Pat. No. 4,322,433) are a few examples of compounds which have shown an ability to enhance the absorption of various compounds.

The pharmaceutical compositions of the invention can also be intended for administration with aerosolized formulation to target areas of a patient's respiratory tract.

Devices and methodologies for delivering aerosolized bursts of a formulation of a drug is disclosed in U.S. Pat. No. 5,906,202. Formulations are preferably solutions, e.g. aqueous solutions, ethanoic solutions, aqueous/ethanoic solutions, saline solutions, colloidal suspensions and microcrystalline suspensions. For example aerosolized particles comprise the active ingredient mentioned above and a carrier, (e.g., a pharmaceutically active respiratory drug and carrier) which are formed upon forcing the formulation through a nozzle which nozzle is preferably in the form of a flexible porous membrane. The particles have a size which is sufficiently small such that when the particles are formed they remain suspended in the air for a sufficient amount of time such that the patient can inhale the particles into the patient's lungs.

The invention encompasses the systems described in U.S. Pat. No. 5,556,611:
liquid gas systems (a liquefied gas is used as propellent gas (e.g. low-boiling FCHC or propane, butane) in a pressure container,
suspension aerosol (the active substance particles are suspended in solid form in the liquid propellent phase),
pressurized gas system (a compressed gas such as nitrogen, carbon dioxide, dinitrogen monoxide, air is used.

Thus, according to the invention the pharmaceutical preparation is made in that the active substance is dissolved or dispersed in a suitable nontoxic medium and said solution or dispersion atomized to an aerosol, i.e. distributed extremely finely in a carrier gas. This is technically possible for example in the form of aerosol propellent gas packs, pump aerosols or other devices known per se for liquid misting and solid atomizing which in particular permit an exact individual dosage.

Therefore, the invention is also directed to aerosol devices comprising the compound as defined above and such a formulation, preferably with metered dose valves.

The pharmaceutical compositions of the invention can also be intended for intranasal administration.

In this regard, pharmaceutically acceptable carriers for administering the compound to the nasal mucosal surfaces will be readily appreciated by the ordinary artisan. These carriers are described in the Remington's Pharmaceutical Sciences” 16th edition, 1980, Ed. By Arthur Osol, the disclosure of which is incorporated herein by reference.

The selection of appropriate carriers depends upon the particular type of administration that is contemplated. For administration via the upper respiratory tract, the composition can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Common intranasal carriers include nasal gels, creams, pastes or ointments with a viscosity of, e.g., from about 10 to about 3000 cps, or from about 2500 to 6500 cps, or greater, may also be used to provide a more sustained contact with the nasal mucosal surfaces. Such carrier viscous formulations may be based upon, simply by way of example, alkylcelluloses and/or other biocompatible carriers of high viscosity well known to the art (see e.g., Remington's, cited supra. A preferred alkylcellulose is, e.g., methylcellulose in a concentration ranging from about 5 to about 1000 or more mg per 100 ml of carrier. A more preferred concentration of methyl cellulose is, simply by way of example, from about 25 to about mg per 100 ml of carrier.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. For nasal administration of solutions or suspensions according to the invention, various devices are available in the art for the generation of drops, droplets and sprays.

A premeasured unit dosage dispenser including a dropper or spray device containing a solution or suspension for delivery as drops or as a spray is prepared containing one or more doses of the drug to be administered and is another object of the invention. The invention also includes a kit containing one or more unit dehydrated doses of the compound, together with any required salts and/or buffer agents, preservatives, colorants and the like, ready for preparation of a solution or suspension by the addition of a suitable amount of water.

Another aspect of the invention is directed to the use of said compound to manufacture a medicament. In other words, the invention embraces a method for treating a disease related to unregulated c-kit transduction comprising administering an effective amount of a compound as defined above to a mammal in need of such treatment.

More particularly, the invention is aimed at a method for treating a disease selected from autoimmune diseases, allergic diseases, bone loss, cancers such as leukemia and GIST, tumor angiogenesis, inflammatory diseases, inflammatory bowel diseases (IBD), interstitial cystitis, mastocytosis, infections diseases, metabolic disorders, fibrosis, diabetes and CNS disorders comprising administering an effective amount a compound depicted above to a mammal in need of such treatment.

The above described compounds are useful for manufacturing a medicament for the treatment of diseases related to unregulated c-kit transduction, including, but not limited to:
neoplastic diseases such as mastocytosis, canine mastocytoma, human gastrointestinal stromal tumor (“GIST”), small cell lung cancer, non-small cell lung cancer, acute myelocytic leukemia, acute lymphocytic leukemia, myelodysplastic syndrome, chronic myelogenous leukemia, colorectal carcinomas, gastric carcinomas, gastrointestinal stromal tumors, testicular cancers, glioblastomas, solid tumors and astrocytomas.
tumor angiogenesis.
metabolic diseases such as diabetes mellitus and its chronic complications; obesity; diabete type II; hyperlipidemias and dyslipidemias; atherosclerosis; hypertension; and cardiovascular disease.
allergic diseases such as asthma, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis and insect bite skin inflammation and blood sucking parasitic infestation.
interstitial cystitis.
bone loss (osteoporosis).
inflammatory diseases such as rheumatoid arthritis, conjunctivitis, rheumatoid spondylitis, osteoarthritis, gouty arthritis and other arthritic conditions.
autoimmune diseases such as multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, as well as proliferative glomerulonephritis.
graft-versus-host disease or graft rejection in any organ transplantation including kidney, pancreas, liver, heart, lung, and bone marrow.
Other autoimmune diseases embraced by the invention active chronic hepatitis and chronic fatigue syndrome.
subepidermal blistering disorders such as pemphigus.
Vasculitis.
melanocyte dysfunction associated diseases such as hypermelanosis resulting from melanocyte dysfunction and including lentigines, solar and senile lentigo, Dubreuilh melanosis, moles as well as malignant melanomas. In this regard, the invention embraces the use of the compounds defined above to manufacture a medicament or a cosmetic composition for whitening human skin.
CNS disorders such as psychiatric disorders, migraine, pain, memory loss and nerve cells degeneracy. More particularly, the method according to the invention is useful for the treatment of the following disorders: Depression including dysthymic disorder, cyclothymic disorder, bipolar depression, severe or “melancholic” depression, atypical depression, refractory depression, seasonal depression, anorexia, bulimia, premenstrual syndrome, post-menopause syndrome, other syndromes such as mental slowing and loss of concentration, pessimistic worry, agitation, self-deprecation, decreased libido, pain including, acute pain, postoperative pain, chronic pain, nociceptive pain, cancer pain, neuropathic pain, psychogenic pain syndromes, anxiety disorders including anxiety associated with hyperventilation and cardiac arrhythmias, phobic disorders, obsessive-compulsive disorder, posttraumatic stress disorder, acute stress disorder, generalized anxiety disorder, psychiatric emergencies such as panic attacks, including psychosis, delusional disorders, conversion disorders, phobias, mania, delirium, dissociative episodes including dissociative amnesia, dissociative fugue and dissociative identity disorder, depersonalization, catatonia, seizures, severe psychiatric emergencies including suicidal behaviour, self-neglect, violent or aggressive behaviour, trauma, borderline personality, and acute psychosis, schizophrenia including paranoid schizophrenia, disorganized schizophrenia, catatonic schizophrenia, and undifferentiated schizophrenia,
neurodegenerative diseases including Alzheimer's disease, Parkinson's disease, Huntington's disease, the prion diseases, Motor Neurone Disease (MND), and Amyotrophic Lateral Sclerosis (ALS).
substance use disorders as referred herein include but are not limited to drug addiction, drug abuse, drug habituation, drug dependence, withdrawal syndrome and overdose.
Cerebral ischemia
Fibrosis
Duchenne muscular dystrophy

Regarding mastocytosis, the invention contemplates the use of the compounds as defined above for treating the different categories which can be classified as follows:

The category I is composed by two sub-categories (IA and 13). Category IA is made by diseases in which mast cell infiltration is strictly localized to the skin. This category represents the most frequent form of the disease and includes: i) urticaria pigmentosa, the most common form of cutaneous mastocytosis, particularly encountered in children, ii) diffuse cutaneous mastocytosis, iii) solitary mastocytoma and iv) some rare subtypes like bullous, erythrodermic and teleangiectatic mastocytosis. These forms are characterized by their excellent prognosis with spontaneous remissions in children and a very indolent course in adults. Long term survival of this form of disease is generally comparable to that of the normal population and the translation into another form of mastocytosis is rare. Category IB is represented by indolent systemic disease (SM) with or without cutaneous involvement. These forms are much more usual in adults than in children. The course of the disease is often indolent, but sometimes signs of aggressive or malignant mastocytosis can occur, leading to progressive impaired organ function.

The category II includes mastocytosis with an associated hematological disorder, such as a myeloproliferative or myelodysplastic syndrome, or acute leukemia. These malignant mastocytosis does not usually involve the skin. The progression of the disease depends generally on the type of associated hematological disorder that conditiones the prognosis.

The category III is represented by aggressive systemic mastocytosis in which massive infiltration of multiple organs by abnormal mast cells is common. In patients who pursue this kind of aggressive clinical course, peripheral blood features suggestive of a myeloproliferative disorder are more prominent. The progression of the disease can be very rapid, similar to acute leukemia, or some patients can show a longer survival time.

Finally, the category IV of mastocytosis includes the mast cell leukemia, characterized by the presence of circulating mast cells and mast cell progenitors representing more than 10% of the white blood cells. This entity represents probably the rarest type of leukemia in humans, and has a very poor prognosis, similar to the rapidly progressing variant of malignant mastocytosis. Mast cell leukemia can occur either de novo or as the terminal phase of urticaria pigmentosa or systemic mastocytosis.

The invention also contemplates the method as depicted for the treatment of recurrent bacterial infections, resurging infections after asymptomatic periods such as bacterial cystitis. More particularly, the invention can be practiced for treating FimH expressing bacteria infections such as Gram-negative enterobacteria including E. coli, Klebsiella pneumoniae, Serratia marcescens, Citrobactor freudii and Salmonella typhimurium.

In this method for treating bacterial infection, separate, sequential or concomitant administration of at least one antibiotic selected bacitracin, the cephalosporins, the penicillins, the aminoglycosides, the tetracyclines, the streptomycins and the macrolide antibiotics such as erytliromycin; the fluoroquinolones, actinomycin, the sulfonamides and trimethoprim, is of interest.

In one preferred embodiment, the invention is directed to a method for treating neoplastic diseases such as mastocytosis, canine mastocytoma, human gastrointestinal stromal tumor (“GIST”), small cell lung cancer, non-small cell lung cancer, acute myelocytic leukemia, acute lymphocytic leukemia, myelodysplastic syndrome, chronic myelogenous leukemia, colorectal carcinomas, gastric carcinomas, gastrointestinal stromal tumors, testicular cancers, glioblastomas, and astrocytomas comprising administering a compound as defined herein to a human or mammal, especially dogs and cats, in need of such treatment.

In one other preferred embodiment, the invention is directed to a method for treating allergic diseases such as asthma, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis and insect bite skin inflammation and blood sucking parasitic infestation comprising administering a compound as defined herein to a human or mammal, especially dogs and cats, in need of such treatment.

In still another preferred embodiment, the invention is directed to a method for treating inflammatory diseases such as rheumatoid arthritis, conjunctivitis, rheumatoid spondylitis, osteoarthritis, gouty arthritis and other arthritic conditions comprising administering a compound as defined herein to a human in need of such treatment.

In still another preferred embodiment, the invention is directed to a method for treating autoimmune diseases such as multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, as well as proliferative glomerulonephritis comprising administering a compound as defined herein to a human in need of such treatment.

In still another preferred embodiment, the invention is directed to a method for treating graft-versus-host disease or graft rejection in any organ transplantation including kidney, pancreas, liver, heart, lung, and bone marrow comprising administering a compound as defined herein to a human in need of such treatment.

### Example 1

### In vitro TK Inhibition Assays

Procedure

Experiments were performed using purified intracellular domain of c-kit expressed in baculovirus. Estimation of the kinase activity was assessed by the phosphorylation of tyrosine containing target peptide estimated by established ELISA assay.

Experimental Results on Tested Compounds

Result in Table 1 shows the potent inhibitory action of the catalytic activity of c-kit with an IC50<10 μM. Further experiments (not shown) indicates that at least one compound acts as perfect competitive inhibitors of ATP.

### Example 2

### Ex Vivo TK Inhibition Assays

Procedures

C-Kit WT and Mutated C-Kit (JM) Assay

Proliferation Assays

Cells were washed two times in PBS before plating at 5×104 cells per well of 96-well plates in triplicate and stimulated either with hematopoietic growth factors (HGF) or without. After 2 days of culture, 37 Bq (1.78 Tbq/mmol) of [3H] thymidine (Amersham Life Science, UK) was added for 6 hours. Cells were harvested and filtered through glass fiber filters and [3H] thymidine incorporation was measured in a scintillation counter. For proliferation assay, all drugs were prepared as 20 mM stock solutions in DMSO and conserved at −80° C. Fresh dilutions in PBS were made before each experiment. DMSO dissolved drugs were added at the beginning of the culture. Control cultures were done with corresponding DMSO dilutions. Results are represented in percentage by talking the proliferation without inhibitor as 100%.

Cells

Ba/F3 murine kit and human kit, Ba/F3 mkitD27 (juxtamembrane deletion) are derived from the murine IL-3 dependent Ba/F3 proB lymphoid cells. The FMA3 and P815 cell lines are mastocytoma cells expressing endogenous mutated forms of Kit, i.e., frame deletion in the murine juxtamembrane coding region of the receptor-codons 573 to 579. The human leukaemic MC line HMC-1 expresses mutations JM-V560G;

Immunoprecipitation Assays and Western Blotting Analysis

For each assay, 5.106 Ba/F3 cells and Ba/F3-derived cells with various c-kit mutations were lysed and immunoprecipitated as described (Beslu et al., 1996), excepted that cells were stimulated with 250 ng/ml of rmKL. Cell lysates were immunoprecipitated with a rabbit immunserum anti murine KIT, directed against the KIT cytoplasmic domain (Rottapel et al., 1991). Western blot was hybridized either with the 4G10 anti-phosphotyrosine antibody (UBI) or with the rabbit immunserum anti-murine KIT or with different antibodies (described in antibodies paragraph). The membrane was then incubated either with HRP-conjugated goat anti mouse IgG antibody or with HRP-conjugated goat anti rabbit IgG antibody (Immunotech), Proteins of interest were then visualized by incubation with ECL reagent (Amersham).

Experimental Results

The experimental results for various compounds according to the invention using above-described protocols are set forth at Table 2:

TABLE 2

| Target | IC50 (mM) | Compounds |
|---|---|---|
| c-Kit WT | IC50 < 10 mM | 001; 002; 003; 004; 005; 028; 029; 030; 031; |
| | | 032; 033; 034; 35; 036; 038; 039; 040; 041; |
| | | 042; 043; 044; 045; 046; 047; 048; 049; 050; |
| | | 051; 052; 053; 054; 055; 056; 057; 058; 059; |
| | | 060; 061; 062; 0.65; 089; 090; 092; 093; |
| | | 094; 096; 099; 100; 101; 102; 105; 106 |
| c-Kit JM | IC50 < 1 mM | |
| D27 | | |

1. A compound having formula I: 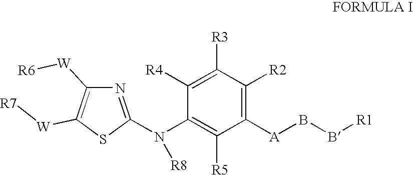
wherein
R⁶ and R⁷ are independently from each other selected from one of the following:
i) H, F, Cl, Br and I;
ii) an alkyl¹ group defined as a linear, branched or cycloalkyl group containing from 1 to 10 carbon atoms, wherein the alkyl¹ group is optionally substituted with one or more heteroatoms selected from F, Cl, Br I, oxygen, and nitrogen, wherein the nitrogen heteroatom is optionally in the form of a pendant basic nitrogen functionality;
trifluoromethyl, carboxyl, cyano, nitro, and formyl;
(iii) an aryl¹ group defined as phenyl or a substituted variant thereof that contains one or more substituents selected from
I, F, Cl and Br;
an alkyl¹ group;
a cycloalkyl, aryl and heteroaryl group optionally substituted with a pendant basic nitrogen functionality,
trifluoromethyl, O-alkyl¹, carboxyl, cyano, nitro, formyl, hydroxy, NH-alkyl¹, N(alkyl¹)(alkyl¹), and amino, wherein each of the NH-alkyl¹, N(alkyl¹)(alkyl¹) and amino substituents is optionally in the form of a basic nitrogen functionality;
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which optionally contains one or more substituents selected from
F, Cl, Br and I;
an alkyl¹ group;
a cycloalkyl, aryl or heteroaryl group optionally substituted with a pendant basic nitrogen functionality,
trifluoromethyl, O-alkyl¹, carboxyl, cyano, nitro, formyl, hydroxy, NH-alkyl¹, N(alkyl¹)(alkyl¹), and amino, wherein each of the NH-alkyl¹, N(alkyl¹)(alkyl¹), and amino substituents is optionally in the form of a basic nitrogen functionality;
(v) trifluoromethyl, carboxyl, cyano, nitro, formyl, hydroxy, N(alkyl¹)(alkyl¹), and amino, wherein each of the N(alkyl¹)(alkyl¹) and amino substituents is optionally in the form of a basic nitrogen functionality;
R⁸ is selected from
(i) hydrogen,
(ii) a linear or branched alkyl group containing from 1 to 10 carbon atoms and optionally substituted with one or more hetereoatoms (selected from F, Cl, Br and I oxygen, and nitrogen, wherein the nitrogen heteroatom is optionally in the form of a pendant basic nitrogen functionality,
(iii) CO—R8, COOR8, CONHR8 or SO2R8 wherein R8 is
a linear or branched alkyl group containing from 1 to 10 carbon atoms and optionally substituted with one or more hetereoatoms selected from F, Cl, Br, I oxygen, and nitrogen, wherein the nitrogen heteroatom is optionally in the form of a pendant basic nitrogen functionality,
an aryl group defined as phenyl or a substituted variant thereof that contains one or more substituents selected from F, Cl, Br I; alkyl groups containing from 1 to 10 carbon atoms and optionally substituted with one or more hetereoatoms selected from F, Cl, Br I; oxygen, and nitrogen, wherein the nitrogen heteroatom is optionally in the form of a pendant basic nitrogen functionality; trifluoromethyl, C₁₋₆ alkyloxy, carboxyl, cyano, nitro, formyl, hydroxy, C₁₋₆ alkylamino, di(C₁₋₆alkyl)amino, and amino, wherein each of the C₁₋₆ alkylamino, di(C₁₋₆alkyl)amino, and amino substituents is optionally in the form of a pendant basic nitrogen functionality; CO—R, COO—R, CONH—R, S02-R, and SO2NH—R, wherein R is a linear or branched alkyl group containing from 1 to 10 carbon atoms and optionally substituted with at least one heteroatom selected from F, Cl, Br I, oxygen, and nitrogen, wherein the nitrogen heteroatom is optionally in the form of a pendant basic nitrogen functionality, or
a heteroaryl group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, or quinolinyl group, the heteroaryl group contains one or more substituents selected from F, Cl, Br, I; alkyl groups containing from 1 to 10 carbon atoms and optionally substituted with one or more hetereoatoms selected from F, Cl, Br, I oxygen, and nitrogen, the latter wherein the nitrogen heteroatom is optionally in the form of a pendant basic nitrogen functionality; trifluoromethyl, C₁₋₆ alkyloxy, carboxyl, cyano, nitro, formyl, hydroxy, C₁₋₆alkylamino, di(C₁₋₆alkyl)amino, amino, wherein each of the C₁₋₆alkylamino, di(C₁₋₆alkyl)amino and amino substituents optionally in the form of a basic nitrogen functionality; CO—R, COO—R, CONH—R, S02-R, and S02NH—R, wherein R is a linear or branched alkyl group containing from 1 to 10 carbon atoms and optionally substituted with at least one heteroatom, selected from F, Cl, Br, I, oxygen, and nitrogen, wherein the nitrogen heteroatom is optionally in the form of a pendant basic nitrogen functionality;
R2, R3, R4 and R5 each independently are selected from hydrogen, F, Cl, Br, I; a linear or branched alkyl group containing from 1 to 10 carbon atoms and optionally substituted with one or more hetereoatoms selected from F, Cl, Br, I oxygen, and nitrogen, wherein the nitrogen heteroatom is optionally in the form of a pendant basic nitrogen functionality; trifluoromethyl, C₁₋₆alkyloxy, amino, C₁₋₆alkylamino, di(C₁₋₆alkyl)amino, carboxyl, cyano, nitro, formyl, hydroxy, CO—R, COO—R, CONH—R, S02-R, and SO2NH—R, wherein R is a linear or branched alkyl group containing from 1 to 10 carbon atoms and optionally substituted with at least one heteroatom selected from F, Cl, Br, I, oxygen, and nitrogen, wherein the nitrogen heteroatom is optionally in the form of a pendant basic nitrogen functionality
A is: CH2, 0, S, S02, CO, or COO,
B is a bond or NH, NCH3, NR*, (CH2)n (n is 0, 1 or 2), 0, S, S02, CO, or COO,
B′ is a bond or NH, NCH3, NR*, (CH2)n (n is 0, 1 or 2), 0, S, SO2, CO or COO;
R* being an alkyl¹, aryl¹ or heteroaryl¹
W is a bond or a linker selected from NH, NHCO, NHCOO, NHCONIH, NHSO2, NHSO2NH, CO, CONH, COO, COCH2, (CH2)n (n is 0, 1 or 2), CH2-CO, CH2COO, CH2-N}I, 0, OCH2, S. SO2, and SO2NH
R¹ is:
a) a linear or branched alkyl group containing from 1 to 10 carbon atoms optionally substituted with at least one heteroatom, selected from I, Cl, Br and F, and/or bearing a pendant basic nitrogen functionality;
b) an aryl or heteroaryl group optionally substituted with an alkyl or aryl group optionally substituted with a heteroatom selected from I, Cl, Br and F or bearing a pendant basic nitrogen functionality;
c) an alkyl¹, aryl¹ or heteroaryl¹.

2. The compound according to claim 1, wherein R6 is (iv), R4 is H or CH3, A-B-B′ is CONH.

3. The compound according to claim 1 that has formula II: 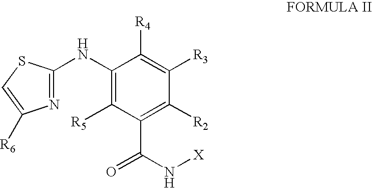
wherein X is R or NRR′ and wherein R and R′ are independently selected from
H,
an aryl, a heteroaryl, an alkyl, or a cycloalkyl group optionally substituted with at least one heteroatom selected from F, I, Cl and Br and optionally bearing a pendant basic nitrogen functionality;
an aryl, a heteroaryl, an alkyl or a cycloalkyl group substituted with an aryl,
a heteroaryl, an alkyl or a cycloalkyl group optionally substituted with at least one heteroatom, selected from F, I, Cl and Br and optionally bearing a pendant basic nitrogen functionality,
R² is hydrogen, halogen; a linear or branched alkyl group containing from 1 to 10 carbon atoms; trifluoromethyl or alkoxy;
R³ is hydrogen, halogen; a linear or branched alkyl group containing from 1 to 10 carbon atoms; trifluoromethyl or alkoxy;
R⁴ is hydrogen, halogen; a linear or branched alkyl group containing from 1 to 10 carbon atoms; trifluoromethyl or alkoxy;
R⁵ is hydrogen, halogen; linear or branched alkyl group containing from 1 to 10 carbon atoms; trifluoromethyl or alkoxy;
R⁶ is one of the following:
(i) an aryl group defined such as phenyl or a substituted variant thereof containing one or more substituents selected from halogen, alkyl groups containing from 1 to 10 carbon atoms, trifluoromethyl, and alkoxy;
(ii) a 2, 3, or 4-pyridyl group, which of optionally contains one or more substituents selected from halogen, alkyl groups containing from 1 to 10 carbon atoms, trifluoromethyl and alkoxy,
(iii) a five-membered ring aromatic heterocyclic group selected from 2-thienyl, 3-thienyl, 2-thiazolyl, 4-thiazolyl, and 5-thiazolyl, wherein the five-membered ring aromatic group optionally contains one or more substituents selected from halogen, an alkyl group containing from 1 to 10 carbon atoms, trifluoromethyl, and alkoxy.
iv) H, I, F, Cl, Br; NH2, N02 or S02-R, wherein R is a linear or branched alkyl group containing 1 to 10 carbon atoms, and optionally substituted with at least one heteroatom, selected from I, Cl, Br and F, and/or bearing a pendant basic nitrogen functionality.

4. The compound according to claim 1, wherein R1 and X, respectively, is a substituted alkyl, aryl or heteroaryl group bearing a pendant basic nitrogen functionality represented for example by the structures a to m shown below, wherein the wavy line and the arrow line correspond to the point of attachment to core structure of formula I or II

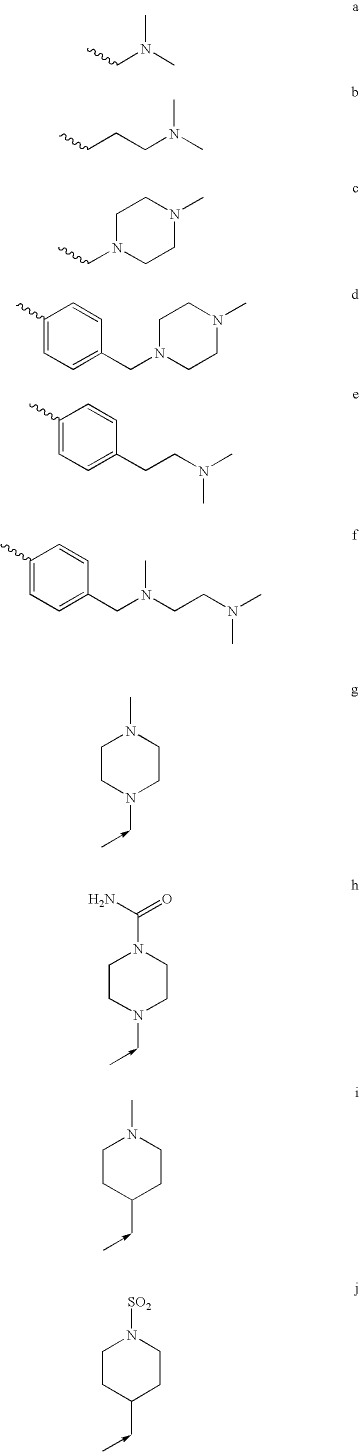

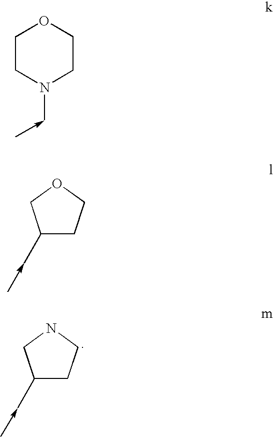

5. The compound according to claim 4, wherein the arrow is a point of attachment to the core structure via a phenyl group.

6. The compound according to claim 1, wherein R6 is a 3-pyridyl group (cf. structure g below), or a 4-pyridyl group (cf. structure h below), the wavy line in each of the structures g and h correspond to the point of attachment to the core structure of formula I or II:

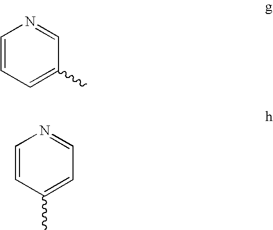

7. The compound according to claim 3 of that has formula II-3: 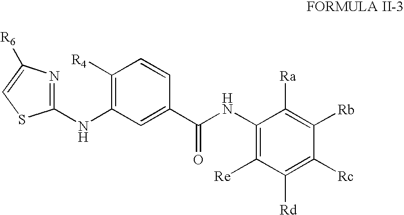
wherein Ra, Rb, Rc, Rd, Re are independently selected from H a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, selected from I, Cl, Br and F or bearing a pendant basic nitrogen functionality; a cycloalkyl, an aryl or heteroaryl group optionally substituted with a cycloakyl, an aryl or heteroaryl group optionally substituted with a heteroatom, selected from I, Cl, Br and F or bearing a pendant basic nitrogen functionality;
a —S02-R groups wherein R is an ailcyl, cycloalkyl, aryl or heteroaryl optionally substituted with a heteroatom, selected from I, Cl, Br and F or bearing a pendant basic nitrogen functionality, or a —CO—R or a —C0-NRR′ group, wherein R and R′ are independently selected from H, an alkyl, a cycloalkyl, an aryl or heteroaryl group optionally substituted with at least one heteroatom, selected from I, Cl, Br and F, and/or bearing a pendant basic nitrogen functionality;
I, Cl, Br and F;
a NRR′ group; where R and R′ are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom selected from I, Cl, Br and F or bearing a pendant basic nitrogen functionality;
an OR group, where R is H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, selected from I, Cl, Br and F or bearing a pendant basic nitrogen functionality; a cycloalkyl, an aryl or heteroaryl group optionally substituted with a cycloalkyl, an aryl or heteroaryl group optionally substituted with a heteroatom selected from I, Cl, Br and F or bearing a pendant basic nitrogen functionality; a —S02-R′ group wherein R′ is an alkyl, cycloalkyl, aryl or heteroaryl optionally substituted with a heteroatom, selected from I, Cl, Br and F or beating a pendant basic nitrogen functionality;
a NRaCORb group, where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom selected from I, Cl, Br and F or bearing a pendant basic nitrogen functionality; a cycloalkyl, an aryl or heteroaryl group optionally substituted with a cycloalkyl, an aryl or heteroaryl group optionally substituted with an heteroatom selected from I, Cl, Br and F or bearing a pendant basic nitrogen functionality,
a NRaCONRbRc group where Ra and Rb are H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom selected from I, Cl, Br and F or bearing a pendant basic nitrogen functionality; a cycloalkyl, an aryl or heteroaryl group optionally substituted with a cycloalkyl, an aryl or heteroaryl group optionally substituted with an heteroatom, selected from I, Cl, Br and F or bearing a pendant basic nitrogen functionality;
a COOR, where R is a linear or branched alkyl group containing from 1 to 10 carbon atoms atoms optionally substituted with at least one heteroatom and/or bearing a pendant basic nitrogen functionality; a cycloalkyl, an aryl or heteroaryl group optionally substituted with at least one heteroatom selected from I, Cl, Br and F, and/or bearing a pendant basic nitrogen functionality or a cycloalkyl, an aryl or heteroaryl group substituted by an alkyl, a cycloalkyl, an aryl or heteroaryl group optionally substituted with an heteroatom, selected from I, Cl, Br and F, and/or bearing a pendant basic nitrogen functionality;
a CONRaRb, where Ra and Rb are a hydrogen or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with at least one heteroatom selected from I, Cl, Br and F, and/or bearing a pendant basic nitrogen functionality; a cycloalkyl, an aryl or heteroaryl group substituted with an alkyl, a cycloalkyl, an aryl or heteroaryl group optionally substituted with a heteroatom selected from I, Cl, Br and F, and/or bearing a pendant basic nitrogen functionality;
an NHCOOR, where R is a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with at least one heteroatom selected from I, Cl, Br and F, and/or bearing a pendant basic nitrogen functionality; cycloalkyl, an aryl or heteroaryl group substituted with an alkyl, a cycloalkyl, an aryl or heteroaryl group optionally substituted with a heteroatom selected from I, Cl, Br and F, and/or bearing a pendant basic nitrogen functionality;
an OSO₂R, where R is a linear or branched alkyl group containing from 1 to 10 carbon atoms atoms optionally substituted with at least one heteroatom and/or bearing a pendant basic nitrogen functionality; a cycloalkyl, an aryl or heteroaryl group optionally substituted with at least one heteroatom selected from I, Cl, Br and F, and/or beating a pendant basic nitrogen functionality; Of a cycloalkyl, an aryl or heteroaryl group substituted with an alkyl, a cycloalkyl, an aryl or heteroaryl group optionally substituted with an heteroatom selected from I, Cl, Br and F, and/or beating a pendant basic nitrogen functionality;
an NRaOSO₂Rb, where Rb is a linear or branched alkyl group containing from 1 to 10 carbon atoms optionally substituted with at least one heteroatom and/or bearing a pendant basic nitrogen functionality; Ra is a linear or branched alkyl group containing from 1 to 10 carbon atoms optionally substituted with at least one heteroatom and/or bearing a pendant basic nitrogen functionality; a hydrogen; a cycloalkyl, an aryl or heteroaryl group optionally substituted with at least one heteroatom, selected from I, Cl, Br and F, and/or bearing a pendant basic nitrogen functionality; a cycloalkyl, an aryl or heteroalkyl group substituted with an alkyl, a cycloalkyl, an aryl or heteroaryl group optionally substituted with a heteroatom selected from I, Cl, Br and F, and/or bearing a pendant basic nitrogen functionality;
a CN group
a trifluoromethyl group
R⁴ is hydrogen, halogen, a linear or branched alkyl group containing from 1 to 10 carbon atoms, trifluoromethyl or alkoxy;
R⁶ is one of the following:
(i) an aryl group defined as phenyl or a substituted variant thereof containing one or more substituents selected from halogen, alkyl groups containing from 1 to 10 carbon atoms, trifluoromethyl, and alkoxy;
(ii) a 2, 3, or 4-pyridyl group, which optionally contains one or more substituents selected from halogen, alkyl groups containing from 1 to 10 carbon atoms, trifluoromethyl and alkoxy,
(iii) a five-membered ring aromatic heterocyclic group selected from 2-thienyl, 3-thienyl, 2-thiazolyl, 4-thiazolyl, and 5-thiazolyl, that optionally contains one or more substituents selected from halogen, an alkyl group containing from 1 to 10 carbon atoms, trifluoromethyl, and alkoxy,
iv) H, I, F, Cl, Br; NH2, N02 or S02-R, wherein R is a linear or branched alkyl group containing 1 to 10 carbon atoms, and optionally substituted with at least one heteroatom, selected from I, Cl, Br and F, and/or bearing a pendant basic nitrogen functionality.

8. The compound according to claim 7 selected from the group consisting of
N-(2-Fluoro-3-trifluoromethyl-phenyl)-4-methyl-3-(4-pyridin-4-yl-thiazol-2-ylamino)-benzamide, N-(3-Fluoro-phenyl)-4-methyl-3-(4-pyridin-4-yl-thiazol-2-ylamino)-benzamide, 4-Methyl-3-(4-pyridin-4-yl-thiazol-2-ylamino)-N-(3-trifluoromethyl-phenyl)-benzamide, 4-Methyl-N-(4-methyl-3-trifluoromethyl-phenyl)-3-(4-pyridin-4-yl-thiazol-2-ylamin)-benzamide, N-(2-Fluoro-5-trifluoromethyl-phenyl)-4-methyl-3-(4-pyridin-4-yl-thiazol-2-ylamino)-benzamide, N-(4-Cyano-phenyl)-4-methyl-3-(4-pyridin-4-yl-thiazol-2-ylamino)-benzamide, N-(4-Fluoro-phenyl)-4-methyl-3-(4-pyridin-4-yl-thiazol-2-ylamino)-benzamide, N-(3-Fluoro-4-methyl-phenyl)-4-methyl-3-(4-pyridin-4-yl-thiazol-2-ylamino)-benzamide, N-(4-tert-Butyl-phenyl)-4-methyl-3-(4-pyridin-4-yl-thiazol-2-ylamino)-benzamide, N-3-Cyano-phenyl)-4-methyl-3-(4-pyridin-4-yl-thiazol-2-ylamino)-benzamide, N-(3-Cyano-4-methyl-phenyl)-4-methyl-phenyl)-4-methyl-3-(4-pyridin-4-yl-thiazol-2-ylamino)-benzamide, N-(3-Bromo-phenyl)-4-methyl-3-(4-pyridin-4-yl-thiazol-2-ylamino)-benzamide, N-(3-Bromo-4-methyl-phenyl)-methyl-3-(4-pyridin-4-yl-thiazol-2-ylamino)-benzamide, N-(3,5-Dibromo-4-methyl-phenyl)-4-methyl-3-(4-pyridin-4-yl-thiazol-2-ylamino)-benzamide, N-(3-Chloro-phenyl)-4-methyl-3-(4-pyridin-4-yl-thiazol-2-ylamino)-benzamide, N-(3-Chloro-4-methyl-phenyl)-4-methyl-3-(4-pyridin-4-yl-thiazol-2-ylamino)-benzamide, N-(3-Methoxy-phenyl)-4-methyl-3-(4-pyridin-4-yl-thiazol-2-ylamino)-benzamide, 4-Methyl-3-(4-pyridin-4-yl-thiazol-2-ylamino)-N-m-tolyl-benzamide, N-(4-Fluoro-3-methyl-phenyl)-4-methyl-3-(4-pyridin-4-yl-thiazol-2-ylamino)-benzamide, N-(3-Iodo-4-methyl-phenyl)-4-methyl-3-(4-pyridin-4-yl-thiazol-2-ylamino)-benzamide, 4-Methyl-N-(3-nitro-phenyl)-3-(4-pyridin-4-yl-thiazol-2-ylamino)-benzamide, 4-Methyl-3-(4-pyridin-4-yl-thiazol-2-ylamino)-N-p-tolyl-benzamide, 4-Methyl-N-phenyl-3-(4-pyridin-4-yl-thiazol-2-ylamino)-benzamide, N-(3,4-Dimethyl-phenyl-phenyl)-4-methyl-3-(4-pyridin-4-yl-thiazol-2-ylamino)-benzamide, 4-Methyl-3-(4-pyridin-4-yl-thiazol-2-ylamino)-N-(3-trifluoromethoxy-phenyl)-benzamide, N-(3,4-dicyano-phenyl)-4-methyl-3-(4-pyridin-4-yl-thiazol-2-ylamino)-benzamide, N-(2-Fluoro-5-methyl-phenyl)-4-methyl-3-(4-pyridin-4-yl-thiazol-2-ylamino)-benzamide, N-(2,4-Difluoro-phenyl)-4-methyl-3-(4-pyridin-4-yl-thiazol-2-ylamino)-benzamide, N-(4-Cyano-2-fluoro-phenyl)-4-methyl-3-(4-pyridin-4-yl-thiazol-2-ylamino)-benzamide, N-(2-Fluoro-4-methyl-phenyl)-4-methyl-3-(4-pyridin-4-yl-thiazol-2-ylamino)-benzamide, N-(2,4-Difluoro-phenyl)-4-methyl-3-(4-pyridin-3-yl-thiazol-2-ylamino)-benzamide, N-(4-Cyano-2-fluoro-phenyl)-4-methyl-3-(4-pyridin-3-yl-thiazol-2-ylamino)-benzamide, N-(2-Fluoro-4-methyl-phenyl)-4-methyl-3-(4-pyridin-3-yl-thiazol-2-ylamino)-benzamide, N-(4-Cyano-phenyl)-4-methyl-3-)4-pyridin-3-yl-thiazol-2-ylamino)-benzamide, N-(f-Fluoro-phenyl)-4-methyl-3-(4-pyridin-3-yl-thiazol-2-ylamino)-benzamide, 4-Methyl-3-(4-pyridin-3-yl-thiazol-2-ylamino)-N-m-tolyl-benzamide, 4-Methyl-3-(4-pyridin-3-yl-thiazol-2-ylamino)-N-(3-trifluoromethyl-phenyl)-benzamide, 4-Methyl-N-(4-methyl-3-methyl-3-trifluoromethyl-phenyl)-3-(4-pyridin-3-yl-thiazol-2-ylamino)-benzamide, N-(2-Fluoro-3-trifluoromethyl-phenyl)-4-methyl-3-(4-pyridin-3-yl-thiazol-2-ylamino)-benzamide, N-(4-Cyano-3-trifluoromethyl-phenyl)-4-methyl-3-(4-pyridin-3-yl-thiazol-2-ylamino)-benzamide, N-(4-Cyano-3-methyl-phenyl)-4-methyl-3-(4-pyridin-3-yl-thiazol-2-ylamino)-benzamide, 4-Methyl-N-[4-(4-methyl-piperazin-1-ylmethyl)-3-trifluoromethyl-phenyl]-3-(4-pyridin-4-yl-thiazol-2-ylamino)-benzamide, 4-Methyl-N-{4-[1-(4-methyl-piperazin-1-yl)-ethyl]-phenyl}-3-(4-pyridin-3-yl-thiazol-2-ylamino)-benzamide, N-(3-Dimethylamino-phenyl)-4-methyl-3-(4-pyridin-4-yl-thiazol-2-ylamino)-benzamide, and N-(3-Dimethylamino-phenyl)-4-methyl-3-(4-pyridin-3-yl-thiazol-2-ylamino)-benzamide.

9. A pharmaceutical composition comprising the compound according to claim 1.

10. The pharmaceutical composition according to claim 9 which is suitable for oral administration.

11. A dermopharmaceutic or cosmetic composition for topical administration of the compound according to claim 1.

12. A veterinary composition comprising the compound according to claim 1.

13-14. (canceled)

15. A method for treating a disease selected from autoimmune diseases, allergic diseases, bone loss, cancers, tumor angiogenesis, inflammatory diseases, inflammatory bowel diseases (IBD), interstitial cystitis, mastocytosis, infections diseases, metabolic disorders, fibrosis, diabetes and CNS disorders, comprising
administering to a subject in need thereof the compound according to claim 1.
